(19) United States
(12) Patent Application Publication
(10) Pub. No.: US 2020/0359881 A1
(43) Pub. Date: Nov. 19, 2020

(54) ENDOSCOPE SYSTEM

(71) Applicant: Covidien LP, Mansfield, MA (US)

(72) Inventors: Meir Rosenberg, Newton, MA (US); Dwight Meglan, Westwood, MA (US)

(21) Appl. No.: 16/638,201

(22) PCT Filed: Aug. 14, 2018
(86) PCT No.: PCT/US2018/046643

§ 371 Date: Feb. 11, 2020

Related U.S. Application Data
(60) Provisional application No. 62/546,160, filed on Aug. 16, 2017.

(51) Int. Cl. A61B 1/05; A61B 1/06; A61B 1/00

(52) U.S. Cl. CPC A61B 1/05; A61B 1/0615; A61B 1/0052; A61B 1/00009; A61B 1/00006

(57) ABSTRACT

An endoscope includes a tube and a pair of image sensors. The tube includes a proximal portion, and a distal portion pivotably coupled to the proximal portion. The distal portion defines a longitudinal axis. The image sensors are disposed in a linear array along the longitudinal axis defined by the distal portion.

### BACKGROUND

Endoscopes are introduced through an incision or a natural body orifice to observe internal features of a body. Conventional endoscopes include a light transmission pathway, including a fiber guide, for transmitting light from an external light source through the endoscope to illuminate the internal features of the body. Conventional endoscopes also include an image retrieval pathway for transmitting images of these internal features back to an eyepiece or external video system for processing and display on an external monitor.

### SUMMARY

In one aspect of the present disclosure, an endoscope is provided and includes a tube and a pair of image sensors. The tube includes a proximal portion, and a distal portion pivotably coupled to the proximal portion. The distal portion defines a longitudinal axis. The image sensors are disposed in a linear array along the longitudinal axis defined by the distal portion.

In some embodiments, the pair of image sensors may be secured to the distal portion of the tube.

It is envisioned that the distal portion may be pivotable relative to the proximal portion between a first position, in which the longitudinal axis of the distal portion is parallel relative to a longitudinal axis defined by the proximal portion, and a second position, in which the longitudinal axis of the distal portion is non-parallel relative to the longitudinal axis defined by the proximal portion. The endoscope may further include an actuation mechanism having a distal portion coupled to the distal portion of the tube such that movement of the actuation mechanism pivots the distal portion of the tube between the first and second positions.

It is contemplated that the proximal and distal portions of the tube may each define oblique cutouts. The oblique cutouts may together define a sector-shaped opening in the tube when the tube is in a linear configuration. The sector-shaped opening may be configured to allow the distal portion of the tube to articulate relative to the proximal portion of the tube between the linear configuration and a non-linear configuration.

In another aspect of the present disclosure, an endoscope is provided and includes an elongated body, and a pair of sensor assemblies. The elongated body includes a proximal portion and a pair of distal portions pivotably coupled to the proximal portion. The pair of sensor assemblies are secured to the pair of distal portions of the elongated body.

In some embodiments, the elongated body may include a first tube and a second tube disposed in parallel relation to the first tube. The first tube has a proximal portion defining a longitudinal axis, and a first distal portion of the pair of distal portions pivotably coupled to the proximal portion of the first tube. The second tube has a proximal portion defining a longitudinal axis, and a second distal portion of the pair of distal portions pivotably coupled to the proximal portion of the second tube.

It is contemplated that the pair of sensor assemblies may include a first sensor assembly secured to the first distal portion of the first tube, and a second sensor assembly secured to the second distal portion of the second tube.

It is envisioned that the first tube may be rotatable about the longitudinal axis of the proximal portion of the first tube, and the second tube may be rotatable about the longitudinal axis of the proximal portion of the second tube. The first tube may be axially movable along the longitudinal axis of the proximal portion of the first tube, and the second tube may be axially movable along the longitudinal axis of the proximal portion of the second tube.

In some aspects, the first and second tubes may be slidable and rotatable relative to one another.

In some embodiments, the pair of sensor assemblies may be coplanar.

It is contemplated that the endoscope may further include a first actuation mechanism and a second actuation mechanism. The first actuation mechanism may include a distal portion coupled to a first distal portion of the pair of distal portions such that movement of the first actuation mechanism pivots the first distal portion relative to the proximal portion. The second actuation mechanism may include a distal portion coupled to a second distal portion of the pair of distal portions such that movement of the second actuation mechanism pivots the second distal portion relative to the proximal portion.

As used herein, the terms parallel and perpendicular are understood to include relative configurations that are substantially parallel and substantially perpendicular up to about + or −10 degrees from true parallel and true perpendicular.

Further details and aspects of exemplary embodiments of the present disclosure are described in more detail below with reference to the appended figures.

### BRIEF DESCRIPTION OF THE DRAWINGS

Embodiments of the present disclosure are described herein with reference to the accompanying drawings, wherein:

FIG. 1 is a front, perspective view illustrating a schematic configuration of an endoscope system;

FIG. 2 is an enlarged, side view of a distal portion of an endoscope of the endoscope system of FIG. 1;

FIG. 3 is a front view of the distal portion of the endoscope of FIG. 2;

FIG. 4A is a side, partial view of a tube of an endoscope in accordance with another embodiment of the present disclosure illustrating the tube in a linear configuration;

FIG. 4B is a side, partial view of the tube of FIG. 4A in a non-linear configuration;

FIG. 5A is a side, partial view of a tube of an endoscope in accordance with another embodiment of the present disclosure illustrating the tube in a linear configuration;

FIG. 5B is a side, partial view of the tube of FIG. 5A in a non-linear configuration;

FIG. 6A is a side, partial view of an endoscope disposed within a cannula in accordance with another embodiment of the present disclosure illustrating first and second tubes of the endoscope in a linear configuration;

FIG. 6B is a side, partial view of the endoscope of FIG. 6A illustrating the tubes in a non-linear configuration;

FIG. 7A is a side, partial view of an endoscope in accordance with another embodiment of the present disclosure illustrating the endoscope in a linear configuration;

FIG. 7B is a side, partial view of the endoscope of FIG. 7A illustrating the endoscope in a non-linear configuration;

FIG. 8A is a side, partial view of an endoscope in accordance with another embodiment of the present disclosure illustrating the endoscope in a linear configuration;

FIG. 8B is a side, partial view of the tube of FIG. 8A illustrating the endoscope in a non-linear configuration; and

FIG. 9 is a schematic illustration of a robotic surgical system for use with an endoscope of the present disclosure.

### DETAILED DESCRIPTION OF EMBODIMENTS

Embodiments of the presently disclosed endoscope and endoscope system is described in detail with reference to the drawings, in which like reference numerals designate identical or corresponding elements in each of the several views. As used herein, the term “distal” refers to that portion of a structure that is closer to a subject, while the term “proximal” refers to that portion of a structure that is farther from the subject. As used herein, the term “subject” refers to a human patient or other animal. The term “clinician” refers to a doctor, nurse, or other care provider and may include support personnel. The term “about” shall be understood as a word of approximation that takes into account relatively little to no variation in a modified term (e.g., differing by less than 2%).

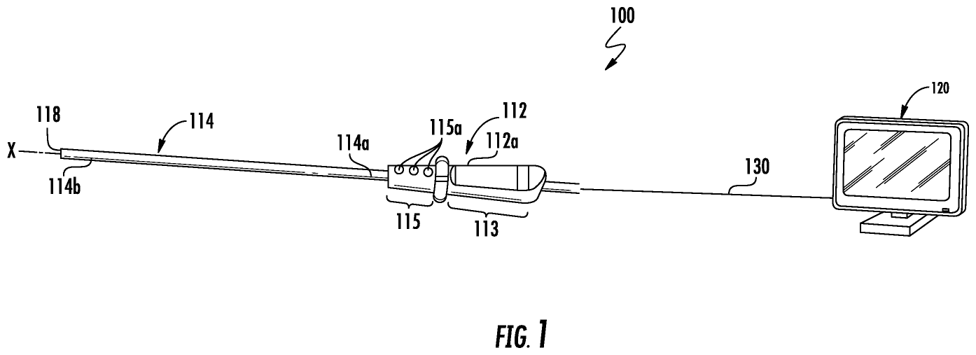

With reference to FIGS. 1-3, an endoscope system 100 of the present disclosure generally includes an endoscope 110, a display 120, and a cable 130 connecting the endoscope 110 and the display 120. Endoscope 110 includes an elongated body or tube 114, and a sensor assembly 140 movably contained within tube 114, as will be described in detail below.

Endoscope 110 includes a handle 112 having tube 114 extending distally therefrom along a longitudinal axis “X.” Tube 114 includes a proximal portion 114a connected to handle 112 and a distal portion 114b terminating at an open distal end or tip 118. Distal portion 114b may be transparent to allow for visual imaging of an outside environment, for example, tissue, by sensor assembly 140. Handle 112 includes a handle housing 112a including a grip portion 113 for handling by a clinician, and a control portion 115 including actuating elements 115a (e.g., buttons, switches etc.) for functional control of endoscope 110.

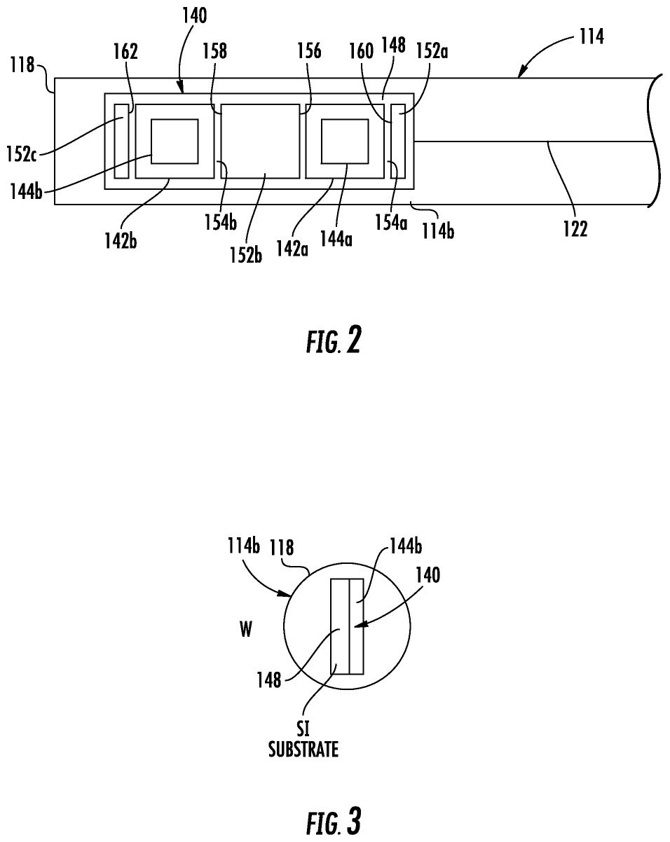

With reference to FIGS. 2 and 3, sensor assembly 140 of endoscope 110 is slidably disposed within distal portion 114b of tube 114 of endoscope 110. Endoscope 110 includes an actuation mechanism 122, for example, a pull wire, coupled to sensor assembly 140 and configured to selectively slide sensor assembly 140 along longitudinal axis “X” of endoscope 110 and to articulate sensor assembly 140 relative to tube 114, as will be described in detail below. Sensor assembly 140 includes a pair of image sensors 142a, 142b longitudinally spaced from one another, and a pair of lenses 144a, 144b corresponding to respective image sensors 142a, 142b. Image sensors 142a, 142b may be charge-coupled devices (CCD), complementary metal-oxide-semiconductors (CMOS), or a hybrid thereof. In embodiments, image sensors 142a, 142b may be highly sensitive, backside illuminated sensors (BSI). In embodiments, the lighting flux required by image sensors 142a, 142b may be up to about 20 lm.

Sensor assembly 140 further includes a substrate 148 and a plurality of lights 152a, 152b, 152c disposed on substrate 148. Substrate 148 is rectangular, and defines a pair of longitudinally spaced apertures or channels 154a, 154b therein. Channels 154a, 154b are illustrated as being circular, but it is contemplated that channels 154a, 154b, along with image sensors 142a, 142b, and/or lenses 144a, 144b may assume a rectangular shape. In particular, the rectangular channels 154a, 154b may have an aspect ratio of about 16:10. In embodiments, substrate 148 may assume any suitable shape.

Image sensors 142a, 142b of sensor assembly 140 are disposed within respective channels 154a, 154b of substrate 148 and lenses 144a, 144b are disposed on respective image sensors 142a, 142b to focus light onto respective image sensors 142a, 142b. In embodiments, lenses 144a, 144b may be focus free lenses. As compared to traditional endoscopes, a focus free lens relies on depth of field to produce sharp images and thus, eliminates the need to determine the correct focusing distance and setting the lens to that focal point.

With continued reference to FIGS. 2 and 3, lights 152a-c of sensor assembly 140 are disposed on substrate 148 and are configured to illuminate an outside environment, for example, tissue, to be viewed by endoscope 110. A first light 152a is disposed on a proximal end of substrate 148, adjacent image sensor 142a. First light 152a has a generally squared configuration with a crescent-shaped cutout 160 defined in a distal end thereof to accommodate image sensor 142a. A second light 152b is disposed between image sensors 142a, 142b. Second light 152b has a generally squared configuration with crescent-shaped cutouts 156, 158 defined in proximal and distal ends thereof to accommodate image sensors 142a, 142b, respectively. A third light 152c is disposed on a distal end of substrate 148, adjacent image sensor 142b. Third light 152c has a generally squared configuration with a crescent-shaped cutout 162 defined in a proximal end thereof to accommodate image sensor 142b.

Lights 152a-c are high efficiency light emitting elements, such as light-emitting diodes (LED). In embodiments, lights 152a-c may have a luminous efficacy of up to about 80 lm/W (lumen/watt). As compared to traditional endoscopes, the lights 152a-c of the present disclosure eliminate the need for the use of an external light source and fiber guide, which can lower the cost of the endoscope system 100, simplify the endoscope system structure, and reduce light consumption and/or light distortion during light transmission.

Heat generation may be managed, for example, by controlling the luminous efficacy of lights 152a-c and the lighting flux required by image sensors 142a, 142b. In embodiments, endoscope 110 of the present disclosure includes high efficiency LED light emitting elements 152a-c and BSI CMOS sensors 142a, 142b. The BSI CMOS sensors 142a, 142b reduce the lighting flux required to get a bright and clear image in a desired body cavity over image sensors utilized in traditional endoscopes. Accordingly, in embodiments where, for example, about 20 lm of lighting flux is required, such as within an abdomen of a patient, the power consumption of LED light emitting elements 152a-c having a luminous efficacy of about 80 lm/W will be about 0.25 W (20 lm/80 lm/W=0.25 W). As about 80% of the power consumption of an LED is typically turned into heat, an LED light emitting elements 152a-c with 0.25 W power consumption would generate no more than about 0.2 W of heat, which is a relatively very small amount of heat that can be controlled by a passive thermal system.

Endoscope system 100 may further include a processor (not shown) configured and designed to capture full high definition raw data from image sensors 142a, 142b of sensor assembly 140 and to transmit the data to an imaging subsystem (not shown) for video processing, including, for example, color conversion, defect correction, image enhancement, H3A (Auto White Balance, Auto Exposure, and Auto Focus), and resizer. The data is then transmitted to a high definition video processing subsystem (not shown) for wrapping of the processed data, and finally to an HDMI output (not shown) for image display on the display device 120 (FIG. 1).

In operation, to view and capture images of an external environment, for example, tissue of a patient, endoscope 110 is inserted within a trocar or cannula (not shown) to gain access within the body of the patient. With endoscope 110 disposed within the body of the patient, light is emitted from one or all of lights 152a-c to illuminate the subject tissue. The light is reflected back toward the lenses 144a, 144b of sensor assembly 140 and directed onto image sensors 142a, 142b of sensor assembly 140, which capture images of the tissue in 3D. Image sensors 142a, 142b ultimately transmit the captured images to display them in 3D on display device 120.

While endoscope 110 is disposed within a body cavity of a patient, sensor assembly 140 may be articulated between a linear configuration and a plurality of non-linear configurations to view or capture images of various tissue areas. In particular, actuation mechanism 122 of endoscope 110 may be actuated to move sensor assembly 140 distally out of distal tip 118 of tube 114 to expose sensor assembly 140 to the tissue area. Actuation mechanism 122 may then be further actuated to articulate sensor assembly 140 relative to tube 114. As such, sensor assembly 140 may be selectively angled, for example, oriented perpendicular, relative to longitudinal axis “X” of tube 114.

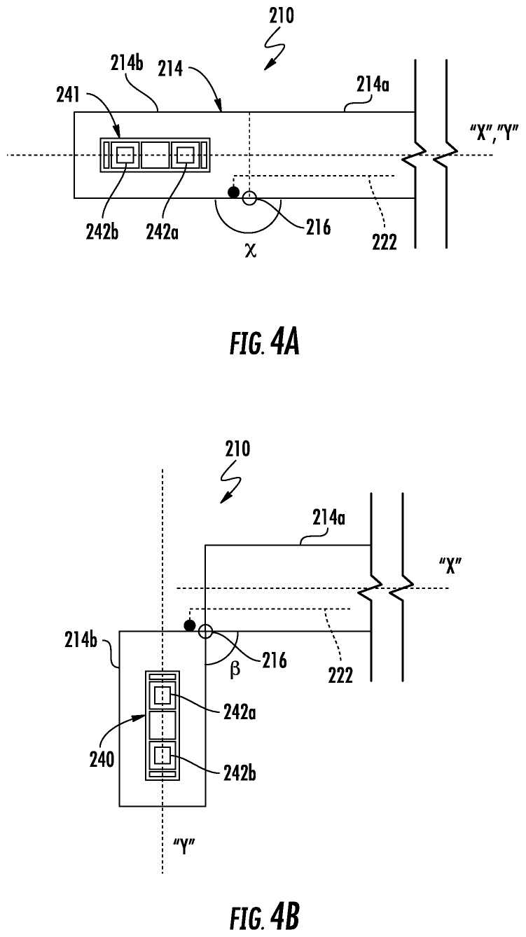

With reference to FIGS. 4A and 4B, another embodiment of an endoscope 210 is illustrated. Endoscope 210 is similar to endoscope 110 described above with reference to FIGS. 2 and 3. Thus, to prevent unnecessary repetition, only selected differences between the embodiments are described. Endoscope 210 generally includes an elongated body or tube 214 and a sensor assembly 240 disposed in tube 214.

Tube 214 of endoscope 210 has a proximal portion 214a and a distal portion 214b. Proximal portion 214a defines a longitudinal axis “X” and distal portion 214b defines a longitudinal axis “Y.” Proximal and distal portions 214a, 214b are pivotably connected to one another via a joint 216, for example, a hinge, such that proximal and distal portions 214a, 214b of tube 214 of endoscope 210 are articulatable relative to one another. Endoscope 210 further includes an actuation mechanism 222, for example, a pull wire, that operably couples distal portion 214b of elongated body 214 to an actuator or trigger (not explicitly shown) of endoscope 210. It is contemplated that distal portion 214b may be articulated relative to proximal portion 214a via actuation of any suitable actuation mechanism.

Sensor assembly 240, which is similar to sensor assembly 140 described above, is disposed within distal portion 214b of tube 214. It is contemplated that distal portion 214b of elongated body 214 is transparent such that light can be transmitted between sensor assembly 240 and an external environment of endoscope 210. In embodiments, sensor assembly 240 may be disposed on an outer surface of distal portion 214b of endoscope 210 rather than within distal portion 214b. Sensor assembly 240 has a generally elongated configuration and defines a longitudinal axis that is parallel with longitudinal axis “Y” of distal portion 214b such that image sensors 242a, 242b thereof are oriented radially away from longitudinal axis “Y.”

In operation, to view and capture images of an external environment, for example, tissue of a patient, endoscope 210 is held in a linear configuration, in which longitudinal axes “X” and “Y” of respective proximal and distal portions 214a, 214b of elongated body 214 are coaxial to define an angle “a” therebetween, wherein “a” is about 180°. While endoscope 210 is in the linear configuration, endoscope 210 is inserted within a trocar or cannula (not shown) to gain access within the body of the patient.

With endoscope 210 disposed within the body of the patient, distal portion 214b may be articulated relative to proximal portion 214a (via actuation of the actuation mechanism 222) to change the angle of longitudinal axis “Y” of distal portion 214b relative to longitudinal axis “X” of proximal portion 214a from angle “α” to a non-linear angle “β.” Since sensor assembly 240 is disposed within distal portion 214b, the orientation of sensor assembly 240 is also changed when distal portion 214b is articulated relative to proximal portion 214a, allowing sensor assembly 240 to capture images of a variety of different areas of the subject tissue.

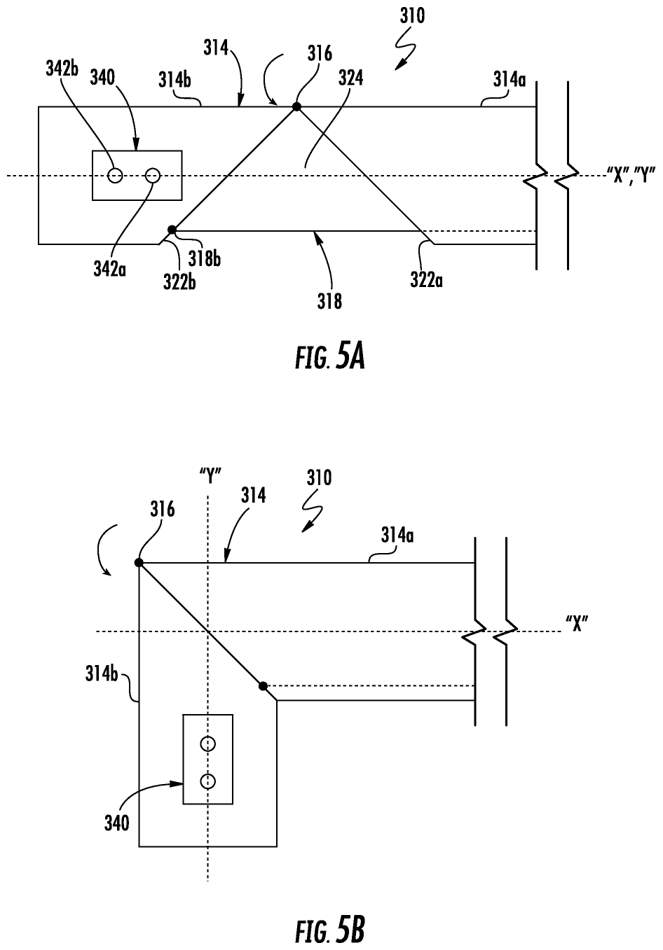

With reference to FIGS. 5A and 5B, another embodiment of an endoscope 310 is illustrated. Endoscope 310 is similar to endoscope 210 described above with reference to FIGS. 4A and 4B. Thus, to prevent unnecessary repetition, only selected differences between the embodiments are described. Endoscope 310 generally includes an elongated body or tube 314 and a sensor assembly 340 disposed in tube 314.

Tube 314 of endoscope 310 has a proximal portion 314a and a distal portion 314b. Proximal portion 314a defines a longitudinal axis “X” and distal portion 314b defines a longitudinal axis “Y.” Proximal and distal portions 314a, 314b are pivotably connected to one another via a joint, for example, a hinge 316, such that proximal and distal portions 314a, 314b of tube 314 of endoscope 310 are articulatable relative to one another. Proximal and distal portions 314a, 314b each define oblique cutouts 322a, 322b such that tube 314 has a sector-shaped opening 324 defined therein when tube 314 is in a linear configuration. Sector-shaped opening 324 allows distal portion 314b to articulate relative to proximal portion 314a about hinge 316, as will be described in further detail below.

Endoscope 310 further includes an actuation mechanism, for example, a pull wire 318, that operably couples distal portion 314b of elongated body 314 to an actuator or trigger (not explicitly shown) of endoscope 310. Pull wire 318 has a proximal portion (not shown) coupled to the actuator or trigger, and a distal portion 318b coupled to distal portion 314b of tube 314. Pull wire 318 is of a rigid construction such that distal translation of pull wire 318 exerts a distal force on distal portion 314b of tube 314 without substantially deforming. In some embodiments, distal portion 314b may be articulated relative to proximal portion 314a via actuation of any suitable actuation mechanism.

Sensor assembly 340, which is similar to sensor assembly 140 described above, is disposed within distal portion 314b of tube 314. It is contemplated that distal portion 314b of elongated body 314 is transparent such that light can be transmitted between sensor assembly 340 and an external environment of endoscope 310. In embodiments, sensor assembly 340 may be disposed on an outer surface of distal portion 314b of endoscope 310 rather than within distal portion 314b. Sensor assembly 340 has a generally elongated configuration and defines a longitudinal axis that is parallel with longitudinal axis “Y” of distal portion 314b such that image sensors 342a, 342b thereof are oriented radially away from longitudinal axis “Y.”

In operation, to view and capture images of an external environment, for example, tissue of a patient, endoscope 310 is held in a linear configuration, in which longitudinal axes “X” and “Y” of respective proximal and distal portions 314a, 314b of elongated body 314 are coaxial to define sector-shaped opening 324. While endoscope 310 is in the linear configuration, endoscope 310 is inserted within a trocar or cannula (not shown) to gain access within the body of the patient.

With endoscope 310 disposed within the body of the patient, the actuator or trigger may be actuated to draw pull wire 318 in a proximal direction. In response to pull wire 318 being moved in a proximal direction within tube 314, distal portion 314b is articulated relative to proximal portion 314a to change the angle of longitudinal axis “Y” of distal portion 314b relative to longitudinal axis “X” of proximal portion 314a. Upon fully articulating proximal portion 314a relative to distal portion 314, oblique cutout 322b of distal portion 314b abuts oblique cutout 322a of proximal portion 314a, thereby closing sector-shaped opening 324 and preventing further articulation. Since sensor assembly 340 is disposed within distal portion 314b, the orientation of sensor assembly 340 is also changed when distal portion 314b is articulated relative to proximal portion 314a, allowing sensor assembly 340 to capture images of a variety of different areas of the subject tissue.

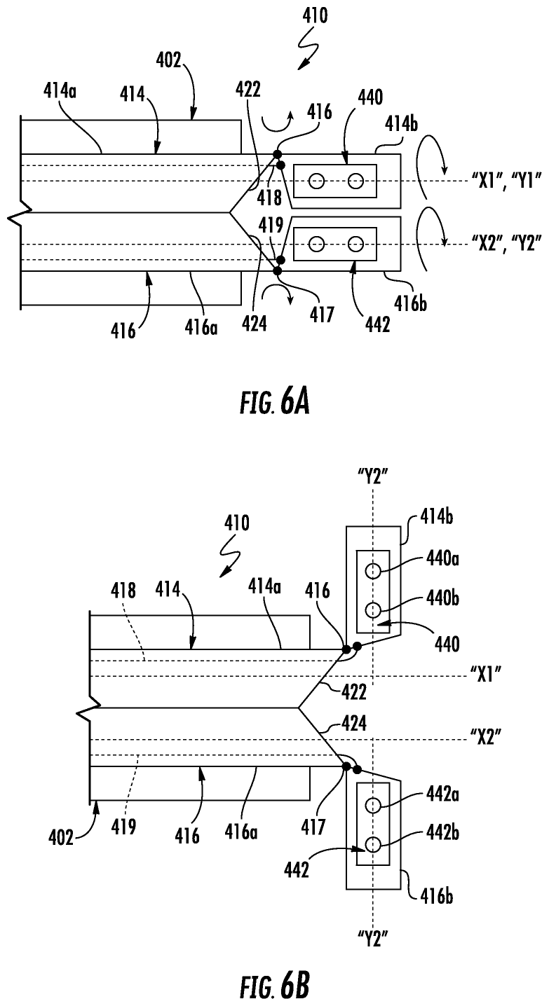

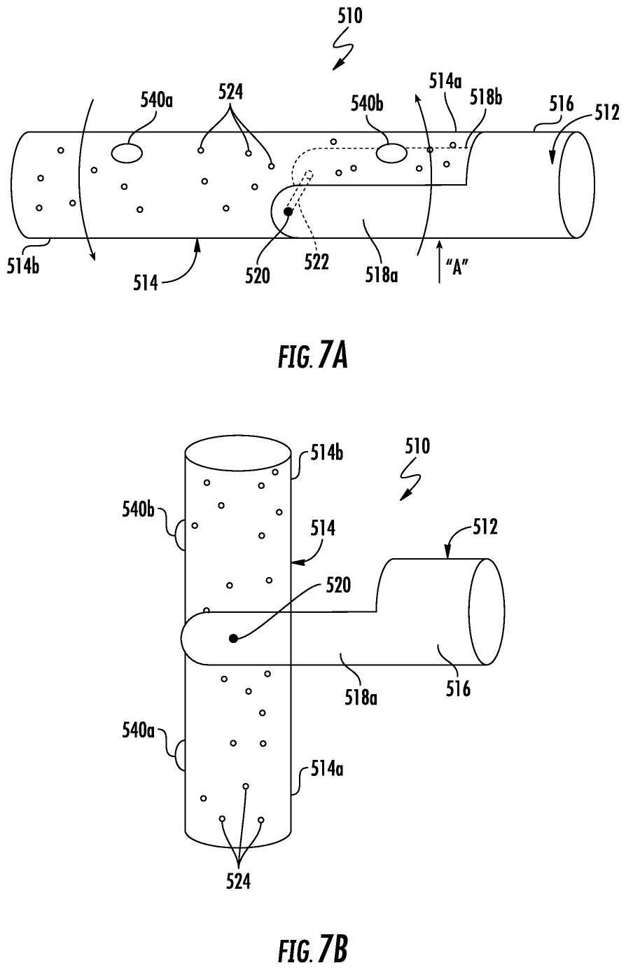

With reference to FIGS. 6A and 6B, another embodiment of an endoscope 410 is illustrated. Endoscope 410 is similar to endoscope 310 described above with reference to FIGS. 5A and 5B. Thus, to prevent unnecessary repetition, only selected differences between the embodiments are described. Endoscope 410 generally includes a pair of elongated bodies or tubes 414, 416 and two sensor assemblies 440, 442 disposed in tubes 414, 416, respectively. Also illustrated is a cannula 402 through which endoscope 410 extends.

First and second tubes 414, 416 of endoscope 410 are disposed in parallel relation to one another. Each of the tubes 414, 416 of endoscope 410 has a proximal portion 414a, 416a and a distal portion 414b, 416b. Proximal portions 414a, 416a define respective longitudinal axes “X1,” “X2,” and distal portions 414b, 416b define respective longitudinal axes “Y1,” “Y2.” It is contemplated that tubes 414, 416 may be independently manipulated. In particular, each of the tubes 414, 416 may be independently rotatable about, and longitudinally movable along, their respective longitudinal axes “X1,” “X2.”

Proximal portions 414a, 416b of tubes 414, 416 are pivotably connected to respective distal portions 414b, 416b of tubes 414, 416 via a joint, for example, a hinge 416, 417, such that distal portions 414b, 416b are articulatable relative to respective proximal portions 414a, 416a, as will be described. Proximal and distal portions 414a, 416a and 414b, 416b of each tube 414, 416 define oblique cutouts 422, 424. Oblique cutouts 422, 424 allow distal portions 414b, 416b to articulate relative to respective proximal portions 414a, 416a about hinges 416, 417.

Each tube 414, 416 may further include an actuation mechanism, for example, pull wires 418, 419, that operably couple distal portions 414b, 416b of tubes 414, 416 to an actuator or trigger (not explicitly shown) of endoscope 410. In some embodiments, endoscope 410 may include only one actuator or trigger for both pull wires 418, 419, or a separate actuator or trigger for each pull wire 418, 419. Pull wires 418, 419 each have a proximal portion (not shown) coupled to the actuator or trigger, and a distal portion coupled to respective distal portions 414b, 416b of tubes 414, 416. Pull wires 418, 419 are of a rigid construction such that distal translation of pull wires 418, 419 exerts a distal force on distal portions 414b, 416b of tubes 414, 416 without substantially deforming. In some embodiments, distal portions 414b, 416 of tubes 414, 416 may be articulated relative to proximal portions 414a, 416a via any suitable actuation mechanism.

Sensor assemblies 440, 442, which are similar to sensor assembly 140 described above, are disposed within distal portions 414b, 416b of tubes 414, 416 respectively. It is contemplated that distal portions 414b, 416b of tubes 414, 416 are transparent such that light can be transmitted between sensor assemblies 440, 442 and an external environment of endoscope 410. In embodiments, sensor assemblies 440, 442 may be disposed on an outer surface of distal portions 414b, 416b of tubes 414, 416 rather than within distal portions 414b, 416b of tubes 414, 416.

Sensor assemblies 440, 442 each have a generally elongated configuration and define a longitudinal axis that is parallel with respective longitudinal axes “Y1,” “Y2” of distal portions 414b, 416b. Sensor assemblies 440, 442 are positioned at a location of distal portions 414b, 416b such that sensor assemblies 440, 442 are coplanar with one another. In some embodiments, sensor assemblies 440, 442 may be disposed on any suitable radial portion of respective distal portions 414b, 416b of tubes 414, 416. For example, sensor assemblies 440, 442 may be disposed on opposing radial portions of distal portions 414b, 416b of respective tubes 414, 416. Sensor assemblies 440, 442 include image sensors 440a, 440b and 442a, 442b that are oriented radially away from longitudinal axes “Y1,” “Y2.”

In operation, to view and capture images of an external environment, for example, tissue of a patient, endoscope 410 is held in a linear configuration, in which longitudinal axes “X1,” “X2” of proximal portions 414a, 414b of tubes 414, 416 are coaxial with respective longitudinal axes “Y1,” “Y2” of distal portions 414b, 416b of tubes 414, 416, as shown in FIG. 6A. While endoscope 410 is in the linear configuration, endoscope 410 is inserted within trocar or cannula 402 to gain access within the body of the patient.

With endoscope 410 disposed within the body of the patient, the actuator or trigger of endoscope 410 may be actuated to draw pull wires 418, 419 in a proximal direction. In response to pull wire 418 being moved in a proximal direction within first tube 414, distal portion 414b of first tube 414 is articulated relative to proximal portion 414a of first tube 414 to change the angle of longitudinal axis “Y1” of distal portion 414b of first tube 414 relative to longitudinal axis “X1” of proximal portion 414a of first tube 414. Similarly, in response to pull wire 419 being moved in a proximal direction within second tube 416, distal portion 416b of second tube 416 is articulated relative to proximal portion 416a of second tube 416 to change the angle of longitudinal axis “Y2” of distal portion 416b of second tube 416 relative to longitudinal axis “X2” of proximal portion 416a of second tube 416.

In accordance with the present disclosure, distal portions 414b, 416b of first and second tubes 414, 416, respectively, are articulated in opposite directions to one another (e.g., substantially symmetrically about a central longitudinal axis of endoscope 410, or cannula 402). As so arranged and configured, when distal portions 414b, 416b of first and second tubes 414, 416 are in the articulated condition, great control and movement of endoscope 410 is achieved, as a whole.

Since sensor assemblies 440, 442 are disposed within distal portions 414b, 416b of tubes 414, 416, the orientation of sensor assemblies 440, 442 is also changed when distal portions 414b, 416b are articulated relative to respective proximal portions 414a, 416a, allowing sensor assemblies 440, 442 to capture images of a variety of different areas of the subject tissue.

While each sensor assembly 440, 442 is described and illustrated as including a respective pair of image sensors 440a, 440b and 442a, 442b, it is contemplated that each sensor assembly 440, 442 may include a single image sensor, with each single image sensor combining to form an image sensor pair or the like. In this manner, individual images or video captured by each single image sensor may be combined (via appropriate image processing) to create a three-dimensional image or video of the operative space.

With reference to FIGS. 7A and 7B, another embodiment of an endoscope 510 is illustrated. Endoscope 510 is similar to endoscope 210 described above with reference to FIGS. 4A and 4B. Thus, to prevent unnecessary repetition, only selected differences between the embodiments are described. Endoscope 510 generally includes a hub 512, an elongated body 514, and first and second image sensors 540a, 540b disposed on elongated body 514.

Hub 512 has a head portion 516 and a pair of arms 518a, 518b extending distally from head portion 516 and parallel to one another. Elongated body 514 is pivotally coupled to and is disposed between arms 518a, 518b of hub 512 via a pin 520. In some embodiments, elongated body 514 may be pivotally coupled to arms 518a, 518b of hub 512 via any suitable connector that enables pivotal movement of elongated body 514 relative to hub 512. Pin 520 extends from first arm 518a, through a central channel 522 defined through elongated body 514, to second arm 518b.

Endoscope 510 may include an actuation mechanism (not shown) that operably couples elongated body 514 to an actuator or trigger (not explicitly shown) of endoscope 510. In some embodiments, endoscope may include a powered actuator, e.g., a pneumatic actuator, a hydraulic actuator, an electric actuator, or other suitable actuator for rotating elongated body 514 relative to hub 512. In some embodiments, endoscope 510 may include gears, belts, friction drives, cables, pulleys, rack and pinions, chains, sprockets, capstans, or any suitable driving mechanism for driving the rotation of elongated body 514 relative to hub 512.

First and second image sensors 540a, 540b of endoscope 510 are disposed on an outer surface of elongated body 514 such that image sensors 540a, 540b are oriented radially away from a longitudinal axis defined by elongated body 514. In some embodiments, image sensors 540a, 540b may be disposed within elongated body 514. First image sensor 540a is disposed on a proximal end portion 514a of elongated body 514 and second image sensor 540b is disposed on a distal end portion 514b of elongated body 514. First and second image sensors 540a, 540b are positioned on elongated body 514 such that first and second image sensors 540a, 540b are aligned with one another and are oriented in the same direction in which elongated body 514 rotates relative to hub 512. Elongated body 514 may include a plurality of lights, for example, LEDs 524, disposed at various locations of elongated body 514.

In operation, to view and capture images of an external environment, for example, tissue of a patient, endoscope 510 is held in a linear configuration (FIG. 7A), in which proximal end portion 514a of elongated body 514 is parallel with arms 518a, 518b of hub 512 and disposed between arms 518a, 518b of hub 512. While endoscope 510 is in the linear configuration, endoscope 510 is inserted within a trocar or cannula (not shown) to gain access within the body of the patient.

With endoscope 510 disposed within the body of the patient, the actuator or trigger of endoscope 510 may be actuated to exert a laterally-oriented force on proximal end portion 514a of elongated body 514, as indicated by arrow “A” in FIG. 7A. In response to this force, elongated body 514 is rotated relative to hub 512 about pivot pin 520. Since first and second image sensors 540a, 540b are disposed on elongated body 514, the orientation of first and second image sensors 540a, 540b is also changed when elongated body 514 is rotated relative to hub 512, allowing first and second image sensors 540a, 540b to capture images of a variety of different areas of the subject tissue. It is contemplated that elongated body 514 may be rotated until elongated body 514 is perpendicular to arms 518a, 518b of hub 512. With elongated body 514 oriented perpendicular relative to arms 518a, 518b of hub 512, image sensors 540a, 540b are oriented in a distal direction.

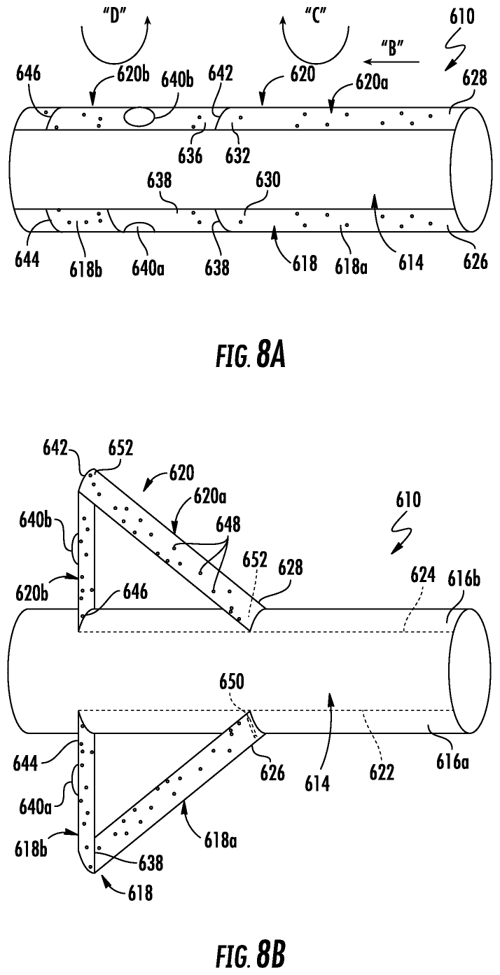

With reference to FIGS. 8A and 8B, another embodiment of an endoscope 610 is illustrated. Endoscope 610 is similar to endoscope 210 described above with reference to FIGS. 4A and 4B. Thus, to prevent unnecessary repetition, only selected differences between the embodiments are described. Endoscope 610 generally includes an elongated body or tube 614, first and second arms 618, 620 coupled to tube 614, and first and second image sensors 640a, 640b disposed on respective first and second arms 618, 620.

Elongated body 614 defines a first track 622 extending longitudinally along a first side 616a thereof, and a second track 624 extending longitudinally along a second side 616b thereof, opposite the first side 616a. First arm 618 of endoscope 610 is operably coupled to first track 622 and second arm 620 of endoscope 610 is operably coupled to second track 624. First and second arms 618, 620 each include a proximal bar 618a, 620a and a distal bar 618b, 620b hingedly coupled to one another. Proximal bar 618a, 620a of each of the first and second arms 618, 620 has a greater length than its respective distal bar 618b, 620b, for example, approximately twice the length of distal bar 618b, 620b. Proximal bar 618a, 620a of each of the first and second arms 618, 620 has a proximal portion 626, 628 that is slidably coupled to respective first and second tracks 622, 624 of elongated body 614, and a distal portion 630, 632 hingedly coupled to a proximal portion 634, 636 of distal bar 618b, 620b, respectively. Hinge or joint 638, 642 of each of the first and second arms 618, 620 allows proximal and distal bars 618a, 618b and 620a, 620b to pivot relative to one another and away from elongated body 614, as will be described below. In some embodiments, proximal and distal bars 618a, 618b and 620a, 620b of each of the first and second arms 618, 620 may be pivotally coupled to one another by any suitable hinge mechanism. For example, hinges 638, 642 may each be a living hinge such that arms 618, 620 are one unitary piece that can be manufactured via printing.

Distal bar 618b, 620b of each of the first and second arms 618, 620 has a distal portion 644 and 646 that is pivotally coupled to elongated body 614 while being axially fixed relative to elongated body 614. As such, distal bars 618b, 620b are pivotable between a first position, in which distal bars 618b, 620b are in abutting engagement with elongated body 614 (FIG. 8A), and a second position, in which distal bars 618b, 620b are perpendicular to elongated body 614 (FIG. 8B).

First image sensor 640a is disposed on an outer surface of distal bar 618b of first arm 618, and second image sensor 640b is disposed on an outer surface of distal bar 620b of second arm 620. First and second image sensors 640a, 640b are positioned on first and second arms 618, 620 such that when first and second arms 618, 620 are in a linear configuration, as shown in FIG. 8A, image sensors 640a, 640b are oriented radially away from a longitudinal axis defined by elongated body 614. First and second arms 618, 620 may include a plurality of lights, for example, LEDs 648, disposed at various locations of first and second arms 618, 620.

Elongated body 614 may include first and second stop members 650, 652 disposed in first and second tracks 622, 624, respectively, of elongated body 614. Stop members 650, 652 are projections or protuberances configured to stop distal sliding of proximal portions 626, 628 of proximal bars 618a, 620a upon proximal portions 626, 628 of proximal bars 618a, 620a advancing into engagement with stop members 650, 652, respectively. It is contemplated that stop members 650, 652 are disposed at a location of elongated body 614 such that upon proximal portions 626, 628 of proximal bars 618a, 620a engaging stop members 650, 652, distal bars 618b, 620b will have attained a perpendicular orientation relative to elongated body 614, as shown in FIG. 8B.

Endoscope 610 may include an actuation mechanism (not shown) that operably couples proximal portions 626, 628 of proximal bars 618a, 620a, respectively, to an actuator or trigger (not explicitly shown) of endoscope 610. The actuator or trigger may be configured to distally advance proximal portions 626, 628 of proximal bars 618a, 620a relative to elongate body 614. In some embodiments, endoscope 610 may include a powered actuator, e.g., a pneumatic actuator, a hydraulic actuator, an electric actuator, or other suitable actuator for moving first and second arms 618, 620 relative to elongated body 614. In some embodiments, endoscope 610 may include gears, belts, friction drives, cables, pulleys, rack and pinions, chains, sprockets, capstans, or any suitable actuation mechanism for driving the movement of first and second arms 618, 620 between the linear configuration and the non-linear, angled configuration.

In operation, to view and capture images of an external environment, for example, tissue of a patient, endoscope 610 is held in a linear configuration (FIG. 8A), in which proximal and distal bars 618a, 618b and 620a, 620b of each of the first and second arms 618, 620 are linear. While endoscope 610 is in the linear configuration, endoscope 610 is inserted within a trocar or cannula (not shown) to gain access within the body of the patient.

With endoscope 610 disposed within the body of the patient, the actuator or trigger of endoscope 610 may be actuated to exert a distally-oriented force on proximal portions 626, 628 of proximal bars 618a, 620a of each of the first and second arms 618, 620, as indicated by arrow “B” in FIG. 8A. It is contemplated that a raised portion or bump (not shown) is disposed on respective tracks 622, 624 beneath hinges 638, 642 to assist in forcing the first and second arms 618, 620 outward upon receiving the distally-oriented force. In response to this force, proximal portion 626, 628 of each of the proximal bars 618a, 620a slides distally along respective first and second tracks 622, 624 causing proximal bar 618a and distal bar 618b of first arm 618 to pivot relative to one another about hinge 638, and likewise causing proximal bar 620a and distal bar 620b of second arm 620 to pivot relative to one another about hinge 642. In particular, since distal portion 644, 646 of distal bars 618b, 620b of each of the first and second arms 618, 620, respectively, are axially fixed relative to elongated body 614 (i.e., not slidable), as proximal portion 626, 628 of proximal bars 618a, 620a slides distally, hinges 638, 642 of first and second arms 618, 620 are forced radially outward. As hinges 638, 642 are forced radially outward, proximal bar 618a, 620a of each of the first and second arms 618, 620 rotates in a first direction, indicated by arrow “C” in FIG. 8A, and distal bar 618b, 620b of each of the first and second arms 618, 620 rotates in a second, opposite direction, indicated by arrow “D” in FIG. 8B.

Distal advancement of proximal portion 626, 628 of proximal bar 618a, 620a of each of the first and second arms 618, 620 along respective first and second tracks 622, 624 is continued until distal bars 618b, 620b are oriented perpendicular to the longitudinal axis defined by elongated body 614. Since first and second image sensors 640a, 640b are disposed on distal bars 618b, 620b, respectively, the orientation of image sensors 640a, 640b is also changed when first and second arms 618, 620 are in the bent configuration (FIG. 8B), allowing image sensors 640a, 640b to capture images of a variety of different areas of the subject tissue. With distal bars 618b, 620b oriented perpendicular relative to the longitudinal axis defined by elongated body 614, image sensors 640a, 640b are oriented in a distal direction.

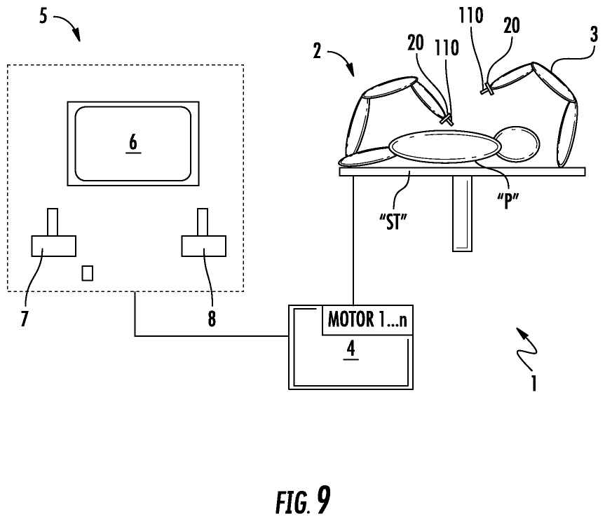

With reference to FIG. 9, the endoscopes of the present disclosure may be configured to be detachably couplable and controllable by a robotic surgical system 1. One exemplary robotic surgical system 1 may generally include a plurality of surgical robotic arms 2, 3 each having an instrument drive unit 20 and an endoscope, for example, endoscope 110, removably attached thereto; a control device 4; and an operating console 5 coupled with the control device 4.

The operating console 5 includes a display device 6, which is set up in particular to display three-dimensional images; and manual input devices 7, 8 by means of which a person, for example, a surgeon, is able to telemanipulate the robotic arms 2,3 in a first operating mode, as known in principle to a person skilled in the art. Each of the robotic arms 2, 3 may be composed of a plurality of members, which are connected through joints. The robotic arms 2, 3 may be driven by electric drives that are connected to the control device 4. The control device (e.g., a computer) 4 is set up to activate the drives, in particular by means of a computer program, in such a way that the robotic arms 2, 3, the attached instrument drive units 20, and thus the endoscope 110, 210, or 310 execute a desired movement according to a movement defined by means of the manual input devices 7, 8. The control device 4 may also be set up in such a way that it regulates the movement of the robotic arms 2, 3 and/or of the drives.

The robotic surgical system 1 is configured for use on a patient “P” lying on a surgical table “ST” to be treated in a minimally invasive manner by means of the endoscope 110. The robotic surgical system 1 may also include more than two robotic arms, the additional robotic arms likewise being connected to the control device 4 and being telemanipulatable by means of the operating console 5. An endoscope may also be attached to the additional robotic arm.

The control device 4 may control a plurality of motors, with each motor configured to drive movement of the robotic arms 2, 3 in a plurality of directions. Further, the control device 4 may control the activation of the instrument drive unit 20 to drive various operations of the endoscope 110.

The robotic surgical system 1 may further include a surgical instrument holder (not shown) configured to be coupled with or to the robotic arm 2, 3. The surgical instrument holder holds the instrument drive unit 20 and the endoscope 110. The surgical instrument holder supports or houses a motor, which receives controls and power from the control device 4 to effect a rotation of an internal motor pack of the instrument drive unit 20, which results in a rotation of the endoscope 110 about a longitudinal axis thereof. The surgical instrument holder may be slidably mounted onto a rail of the robotic arm 2, 3 and moved along the rail via a motor driven chain or belt or the like to adjust a position of the endoscope 110.

For a more detailed description of the construction and operation of a robotic surgical system, reference may be made to U.S. Patent Application Publication No. 2012/0116416, filed on Nov. 3, 2011, entitled “Medical Workstation,” the entire contents of which are incorporated by reference herein.

It will be understood that various modifications may be made to the embodiments described herein. Therefore, the above description should not be construed as limiting, but merely as exemplifications of various embodiments. Those skilled in the art will envision other modifications within the scope and spirit of the claims appended thereto.

What is claimed is:

1. An endoscope, comprising:
a tube including a proximal portion, and a distal portion pivotably coupled to the proximal portion, the distal portion defining a longitudinal axis; and
a pair of image sensors disposed in a linear array along the longitudinal axis defined by the distal portion.

2. The endoscope according to claim 1, wherein the pair of image sensors are secured to the distal portion of the tube.

3. The endoscope according to claim 1, wherein the distal portion is pivotable relative to the proximal portion between a first position, in which the longitudinal axis of the distal portion is parallel relative to a longitudinal axis defined by the proximal portion, and a second position, in which the longitudinal axis of the distal portion is non-parallel relative to the longitudinal axis defined by the proximal portion.

4. The endoscope according to claim 3, further comprising an actuation mechanism having a distal portion coupled to the distal portion of the tube such that movement of the actuation mechanism pivots the distal portion of the tube between the first and second positions.

5. The endoscope according to claim 1, wherein the proximal and distal portions of the tube each define oblique cutouts.

6. The endoscope according to claim 5, wherein the oblique cutouts together define a sector-shaped opening in the tube when the tube is in a linear configuration, the sector-shaped opening configured to allow the distal portion of the tube to articulate relative to the proximal portion of the tube between the linear configuration and a non-linear configuration.

7. An endoscope, comprising:
an elongated body including a proximal portion and a pair of distal portions pivotably coupled to the proximal portion; and
a pair of sensor assemblies secured to the pair of distal portions of the elongated body.

8. The endoscope according to claim 7, wherein the elongated body includes:
a first tube having a proximal portion defining a longitudinal axis, and a first distal portion of the pair of distal portions pivotably coupled to the proximal portion of the first tube; and
a second tube disposed in parallel relation to the first tube and having a proximal portion defining a longitudinal axis, and a second distal portion of the pair of distal portions pivotably coupled to the proximal portion of the second tube.

9. The endoscope according to claim 8, wherein the pair of sensor assemblies includes a first sensor assembly secured to the first distal portion of the first tube, and a second sensor assembly secured to the second distal portion of the second tube.

10. The endoscope according to claim 8, wherein the first tube is rotatable about the longitudinal axis of the proximal portion of the first tube, and the second tube is rotatable about the longitudinal axis of the proximal portion of the second tube.

11. The endoscope according to claim 10, wherein the first tube is axially movable along the longitudinal axis of the proximal portion of the first tube, and the second tube is axially movable along the longitudinal axis of the proximal portion of the second tube.

12. The endoscope according to claim 8, wherein the first and second tubes are slidable and rotatable relative to one another.

13. The endoscope according to claim 7, wherein the pair of sensor assemblies are coplanar.

14. The endoscope according to claim 7, further comprising:
a first actuation mechanism including a distal portion coupled to a first distal portion of the pair of distal portions such that movement of the first actuation mechanism pivots the first distal portion relative to the proximal portion; and
a second actuation mechanism including a distal portion coupled to a second distal portion of the pair of distal portions such that movement of the second actuation mechanism pivots the second distal portion relative to the proximal portion.
